(19) United States
(12) Patent Application Publication
(10) Pub. No.: US 2010/0036191 A1
(43) Pub. Date: Feb. 11, 2010

(54) BRAIN STIMULATION SYSTEMS AND METHODS

(76) Inventors: Timothy J. Walter, Upper Arlington, OH (US); Uma Marar, Blacklick, OH (US)

Correspondence Address: MUETING, RAASCH & GEBHARDT, P.A., P.O. BOX 581336, MINNEAPOLIS, MN, 55458-1336, US

(21) Appl. No.: 12/535,842

(22) Filed: Aug. 5, 2009

Related U.S. Application Data
(60) Provisional application No. 61/086,647, filed on Aug. 6, 2008.

(51) Int. Cl. A61N 2/04

(52) U.S. Cl. 600/14

(57) ABSTRACT

Systems and methods for brain stimulation, e.g., to induce various effects.

The present invention claims the benefit of U.S. Provisional Patent Application Ser. No. 61/086,647, filed Aug. 6, 2008, which is incorporated herein by reference in its entirety.

The present invention relates generally to brain stimulation systems and methods. More specifically, the present invention relates to brain stimulation systems and methods that stimulate portions of a subject's brain to induce various effects.

Transcranial magnetic stimulation (TMS) refers to a noninvasive excitation of neurons in the brain by utilizing magnetic fields to induce electric currents in the brain. An example of TMS may involve placing a treatment coil that generates a magnetic field near a subject's head. The magnetic field may induce an electrical current in the brain causing neurons to fire, which may induce various chemical changes in the brain. Such neuron firings and/or chemical changes may induce various effects in the subject's brain such as, but not limited to, visual information, auditory information, somatosensory information, temporary paralysis, loss of waking consciousness, etc. For instance, TMS on the occipital cortex may induce phosphenes, and TMS on the language cortex may inhibit speech. As described herein, TMS includes all modes of TMS, which may include, e.g., repetitive transcranial magnetic stimulation (rTMS), etc.

TMS has been previously used for multiple purposes. For example, TMS has been used for brain imaging and mapping techniques and pain suppression (United States Patent Application Publication No. 2006/0004422 A1 to Dirk De Riddler, published on Jan. 5, 2006, and entitled “Electrical stimulation system and method for stimulating tissue in the brain to treat a neurological condition”). Further, for example, TMS has been used for functional brain mapping (United States Patent Application Publication No. 2006/0241374 A1 to George et al., published on Oct. 26, 2006, and entitled “Methods and systems for using transcranial magnetic stimulation and functional brain mapping for examining cortical sensitivity, brain communication, and effects of medication”). Still further, for example, TMS has been used for promoting sleep (United States Patent Application Publication No. 2008/0081941 A1 to Giulio Tononi, published on Apr. 3, 2008, and entitled “Method and apparatus for promoting restorative sleep”).

### SUMMARY OF THE INVENTION

In one aspect, the present invention relates generally to brain stimulation systems and methods. For example, the present invention may provide transcranial magnetic stimulation apparatus, position the transcranial magnetic stimulation apparatus proximate a subject's brain, and deliver transcranial magnetic stimulation to at least one portion of the subject's brain to inhibit normal neural processing activity in the at least one portion of the subject's brain, whereby inhibiting the normal neural processing activity in the at least one portion enables a release of a higher association cortex of the subject's brain to induce sensory information generation in the higher association cortex.

In another aspect, the present invention includes a method of stimulating a subject's brain. The method may include: providing stimulation apparatus for delivering transcranial magnetic stimulation; positioning the stimulation apparatus proximate one or more portions of the subject's brain, wherein the stimulation apparatus is located external to the subject's skull; and delivering transcranial magnetic stimulation from the stimulation apparatus to the one or more portions of the subject's brain to inhibit normal neural processing activity in a selected portion of the subject's brain, whereby inhibiting the normal neural processing activity in the selected portion enables a release of a higher association cortex of the subject's brain to induce sensory information generation in the higher association cortex.

In still another aspect, the present invention includes a method of stimulating a subject's brain. The method may include: providing stimulation apparatus for delivering transcranial magnetic stimulation; positioning the stimulation apparatus proximate the subject's primary motor cortex and secondary motor cortex, wherein the stimulation apparatus is located external to the subject's skull; and delivering transcranial magnetic stimulation from the stimulation apparatus to the subject's primary motor cortex and/or secondary motor cortex to inhibit normal neural processing activity of the primary motor cortex and/or secondary motor cortex of the subject's brain, whereby inhibiting the normal neural processing activity of the primary motor cortex and/or secondary motor cortex induces temporary partial paralysis of the subject's body.

In yet another aspect, the present invention includes a method of stimulating a subject's brain. The method may include: providing stimulation apparatus for delivering transcranial magnetic stimulation; positioning the stimulation apparatus proximate a selected portion of the subject's brain, wherein the selected portion of the subject's brain comprises at least one of the frontal association cortex and the dorsolateral prefrontal cortex, wherein the stimulation apparatus is located external to the subject's skull; and delivering transcranial magnetic stimulation from the stimulation apparatus to the selected portion of the subject's brain to inhibit normal neural processing activity of the selection portion of the subject's brain, whereby inhibiting the normal neural processing activity of the selected portion of the subject's brain suppresses the subject's waking consciousness.

In also another aspect, the present invention includes a stimulation system. The stimulation system may include stimulation apparatus for delivering transcranial magnetic stimulation. The stimulation apparatus may be positionable proximate two or more portions of the subject's brain, wherein the two or more portions of the subject's brain comprise two or more of the primary visual cortex, secondary visual cortex, primary auditory cortex, secondary auditory cortex, primary somatosensory cortex, and secondary somatosensory cortex. Further, the stimulation apparatus may be located external to the subject's skull. Still further, the transcranial magnetic stimulation may be deliverable from the stimulation apparatus to two or more selected portions of the two or more portions of the subject's brain to inhibit normal neural processing activity of the two or more selected portions to the subject's brain, whereby inhibiting the normal neural processing activity of the two or more selected portions of the subject's brain enables a release of a higher association cortex of the subject's brain to induce sensory information generation in the higher association cortex. The stimulation system may further include a controller electrically coupled to the stimulation apparatus, wherein the controller includes an input interface to receive input from the subject, and wherein the controller controls the functionality of the stimulation apparatus.

The above summary is not intended to describe each embodiment or every implementation of the present invention. Rather, a more complete understanding of the invention will become apparent and appreciated by reference to the following Detailed Description of Exemplary Embodiments and claims in view of the accompanying figures of the drawing.

### BRIEF DESCRIPTION OF THE DRAWING

FIG. 1 is an illustrative view of an exemplary embodiment of a stimulation system according to the present invention.

FIG. 2 is block diagram of an exemplary method of stimulating a subject's brain according to the present invention.

FIG. 3 is a detailed block diagram of an exemplary method of positioning TMS apparatus proximate one or more portions of a subject's brain as generally illustrated in the method of FIG. 2.

FIG. 4 is a detailed block diagram of another exemplary method of positioning TMS apparatus proximate one or more portions of a subject's brain as generally illustrated in the method of FIG. 2.

FIG. 5 is a detailed block diagram of another exemplary method of positioning TMS apparatus proximate one or more portions of a subject's brain as generally illustrated in the method of FIG. 2.

FIG. 6 is a diagrammatic representation of an exemplary embodiment of a stimulation system according to the present invention.

### DESCRIPTION OF EXEMPLARY EMBODIMENTS

In the following detailed description of illustrative embodiments of the invention, reference is made to the accompanying figures of the drawing which form a part hereof, and in which are shown, by way of illustration, specific embodiments in which the invention may be practiced. It is to be understood that other embodiments may be utilized and structural changes may be made without departing from the scope of the present invention.

As used herein, “a,” “an,” “the,” “at least one,” and “one or more” are used interchangeably. The term “and/or” (if used) means one or all of the listed elements or a combination of any two or more of the listed elements.

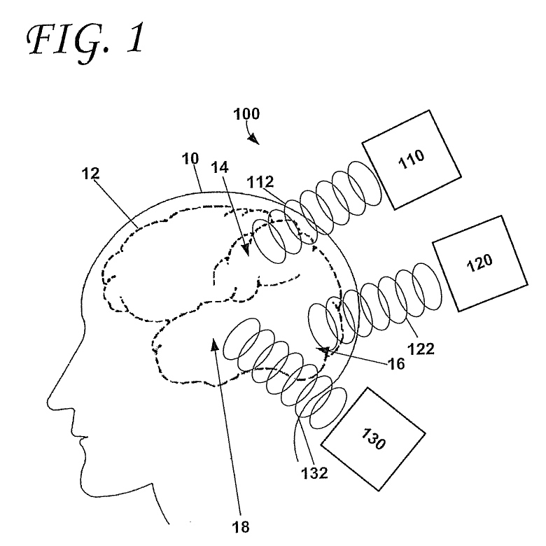

FIG. 1 is an illustrative view of one exemplary embodiment of a stimulation system 100 being used with subject 10. Stimulation system 100 may include transcranial magnetic stimulation apparatus 110, 120, & 130.

As used herein, transcranial magnetic stimulation apparatus may be any apparatus capable of providing transcranial magnetic stimulation to a subject. Examples of some potentially suitable TMS apparatus may include, e.g., a NeuroStar TMS Therapy System from Neuronetics, Inc. The TMS apparatus may include a power supply, a control system, a display system, at least one treatment coil, and/or any other component as would be known by one of ordinary skill in the art. The TMS apparatus may be autonomous, or may be operated by an operator. In at least one embodiment, the subject may operate the TMS apparatus, thereby self-administering the TMS. The TMS apparatus may be stationary or portable (in which case it may include a portable power supply). Further, some components that may included in TMS systems according to the present invention may be described in U.S. Patent Application Pub. No. 2008/0081941 A1, published on Apr. 3, 2008, and entitled “Method and apparatus for promoting restorative sleep.”

Illustrative waves 112, 122, & 132 are shown to represent TMS that may be delivered from the TMS apparatus 110, 120, & 130, respectively, to the brain 12 of the subject 10. Each TMS apparatus 110, 120, & 130 may be positioned proximate a selected portion of the subject's brain 12 to deliver transcranial magnetic stimulation (e.g., represented by waves 112, 122, & 132) to the selected portion of the subject's brain to, e.g., inhibit normal neural processing activity of the selected portions 14, 16, & 18 of the subject's brain 12, whereby inhibiting the normal neural processing activity of the selected portions may enable a release of the higher association cortex of the subject's brain to induce sensory information generation in the higher association cortex.

As described herein, the “higher association cortex” may include Brodmann's areas 5, 7, 19, 22, 37, 39, 40, 42, and/or 49 and/or any other area/portion of a human's brain that may be involved in the higher processing of sensory information as understood by one of ordinary skill in neurophysiology.

Although each TMS apparatus 110, 120, & 130 are shown as being separate, they may be a single, integral unit, or may be multiple interconnected units.

In the embodiment depicted in FIG. 1, TMS apparatus 110 may be positioned proximate the subject's primary and/or secondary somatosensory cortices (e.g., the TMS apparatus may be shaped in an arch to extend from the top of one ear to the other ear of a subject's head to position the TMS apparatus proximate the primary and/or secondary somatosensory cortices) to deliver TMS to the primary and/or secondary somatosensory cortices (e.g., proximate area 14) to, e.g., inhibit normal neural processing activity of the primary and/or secondary somatosensory cortices, whereby inhibiting the normal neural processing activity of the primary and/or secondary somatosensory cortices may enable a release of the higher association cortex to induce internally-generated somatosensory information in higher association cortex of the brain 12 of the subject 10.

As described herein, the “primary somatosensory cortex” may include Brodmann's areas 1, 2, and/or 3 and the “secondary somatosensory cortex” may include Brodmann's areas 5, 7, and/or 40. Further, the both the “primary somatosensory cortex” and “secondary somatosensory cortex” may include any other area/portion of a human's brain that may be involved in the processing of somatosensory information as understood by one of ordinary skill in neurophysiology.

Further, in the embodiment depicted in FIG. 1, TMS apparatus 120 may be positioned proximate the subject's primary and/or secondary visual cortices to deliver TMS to the primary and/or secondary visual cortices (e.g., proximate area 16) to, e.g., inhibit normal neural processing activity of the primary and/or secondary visual cortices, whereby inhibiting the normal neural processing activity of the primary and/or secondary visual cortices may enable a release of the higher association cortex to induce internally-generated visual information in higher association cortex of the brain 12 of the subject 10. In other embodiments, delivering TMS to the subject's primary and/or secondary visual cortices may, e.g., induce the release of dream imagery of recent life events, past events, loose associations of both recent and past events, events of emotional relevance to the subject, unresolved conflicts of the subject, etc.

As described herein, the “primary visual cortex” may include Brodmann's areas 17 and/or 18 and the “secondary visual cortex” may include Brodmann's areas 18, 19, 20, 21, and/or 37. Further, both the “primary visual cortex” and the “secondary visual cortex” may include any other area/portion of a human's brain that may be involved in the processing of visual information as understood by one of ordinary skill in neurophysiology.

Still further, in the embodiment depicted in FIG. 1, TMS apparatus 130 may be positioned proximate the subject's primary and/or secondary auditory cortices to deliver TMS to the primary and/or secondary auditory cortices (e.g., proximate area 18) to, e.g., inhibit normal neural processing activity of the primary and/or secondary auditory cortices, whereby inhibiting the normal neural processing activity of the primary and/or secondary auditory cortices may enable a release of the higher association cortex to induce internally-generated auditory information in higher association cortex of the brain 12 of the subject 10. In other embodiments, delivering TMS to the subject's primary and/or secondary auditory may, e.g., induce self-generated sounds, voices, music, etc.

As described herein, the “primary auditory cortex” and “secondary auditory cortex” may include Brodmann's areas 20, 21, 22, 41, and/or 42, and/or any other area/portion of a human's brain that may be involved in the processing of auditory information as understood by one of ordinary skill in neurophysiology As described herein, when transcranial magnetic stimulation apparatus is positioned proximate a portion of a subject's brain, “proximate a selected portion of a subject's brain” refers to positioning the TMS apparatus external to the subject's skull such that the TMS apparatus is capable of delivering TMS to the selected portion of the subject's brain that it is recited to be “proximate.”

Such brain stimulation may result in a meditation-like state of internally-generated sensory information that may be experienced instead of or in conjunction with normal waking consciousness. Further, such brain stimulation may be used for relaxation, introspection, and/or entertainment value.

Further, a subject using the stimulation system 100 may select one or more of the TMS apparatuses 110, 120, & 130. When only the TMS apparatus 120 (i.e., the TMS apparatus positioned proximate the primary and secondary visual cortices) is activated, only normal neural processing activity of the primary and secondary visual cortices may be inhibited and, as such, only the visual components of the internally-generated imagery may be experienced. Likewise, when only the TMS apparatus 130 (i.e., the TMS apparatus positioned proximate the primary and secondary auditory cortices) is activated, only the auditory components of the internally-generated auditory sensory information may be experienced. Further, when only the TMS apparatus 10 (i.e., the TMS apparatus positioned proximate the primary and secondary somatosensory cortices) is activated, only the somatosensory components of the internally generated sensation may be experienced. This type of selective stimulation may allow a subject to use the device in many situations where the subject may be engaged such as, e.g., commuting on a train or airplane, or during relaxation while some attention to the surrounding environment is required.

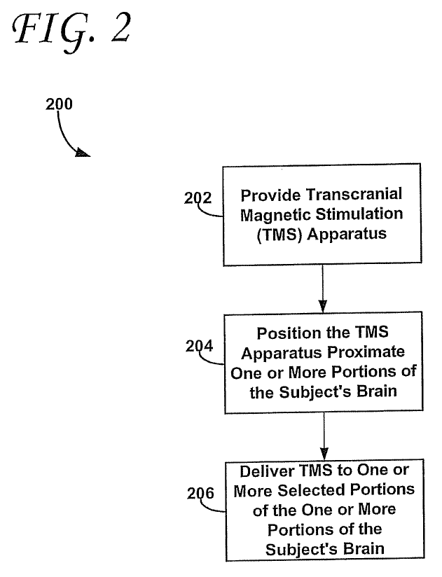

One exemplary embodiment of a method 200 according to the present invention is depicted in FIG. 2. The stimulation method 200 may include providing TMS apparatus 202. The TMS apparatus 202 may be substantially similar to the TMS apparatus 110, 120, & 130 described herein with reference to FIG. 1. As such, for simplicity, further description on the details of TMS apparatus 202 shall not be provided.

The stimulation method 200 may further include positioning the TMS apparatus proximate one or more portions of the subject's brain 204. As described herein, the TMS apparatus may include one or more treatment coils that may deliver magnetic stimulation to, e.g., the subject's brain by producing one or more magnetic fields. Each treatment coil may be positioned proximate a portion of the subject's brain such that TMS may be delivered to that portion of the subject's brain.

For example, the treatment coils may be positioned to stimulate the various cortical and deep brain structures including the primary and secondary visual cortices, the primary and secondary auditory cortices, the primary and secondary somatosensory cortices, the primary and motor cortices, the frontal association cortex, the dorsolateral prefrontal cortex, etc. The portions of the brain may be selected to correspond to areas of the brain that correspond to various brain functions (e.g., the visual cortices corresponds to visual processes) understood by one of ordinary skill in neurophysiology.

Further, the location of the specific cortices within a subject's brain may be different for each subject. As such, the methods and systems described herein may include positioning and adjustment apparatus that may allow positioning and/or adjustment of the TMS apparatus to accommodate different subjects.

The stimulation method 200 may further include delivering TMS to selected portions of the one or more portions of the subject's brain 206. The TMS delivered to a subject may include magnetic fields oscillating at one or more selected frequency ranges, e.g., a frequency of about 3 hertz or more, about 8 hertz or less, or faster or slower frequency ranges. Other variations in the delivery of TMS may include variations in magnetic field strength. For example, the magnetic field strength used in connection with the present invention as measures at the subject's scalp may be about 1 tesla or more, about 2 teslas or more, about 1 tesla or less, about 4 teslas or less, etc. Further, other variations in the delivery of TMS may include variations in waveform morphology and/or variations in the patterns of waveforms through time.

Although TMS apparatus may be positioned proximate one or more portions of the subject's brain, TMS may not be delivered from all of the TMS apparatus, e.g., not all of the TMS apparatus may be selected to deliver TMS even though all of the TMS apparatus may be positioned. For example, TMS apparatus may be positioned proximate the subject's primary and secondary visual, auditory, and somatosensory cortices but the subject and/or operator may elect to only deliver TMS to the subject's primary and secondary visual and auditory cortices.

Further, the TMS may be delivered for a fixed time period (e.g., a fraction of a second). Alternatively, the period of time over which TMS is delivered may vary. For example, the period of time over which TMS is delivered may be based upon real time feedback from the subject to, e.g., achieve the desired result.

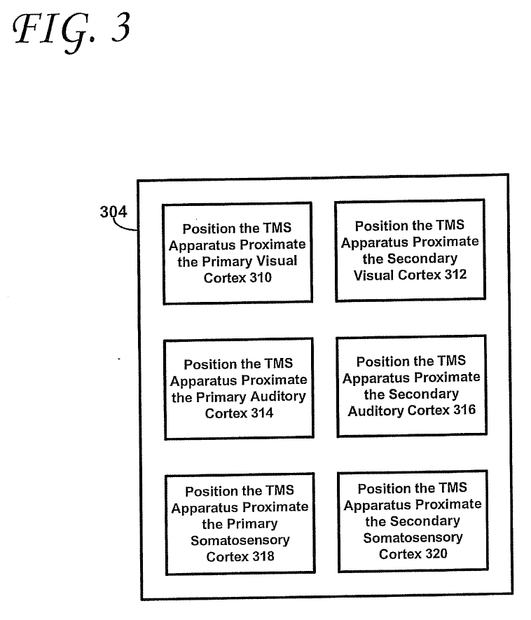

FIG. 3 is a detailed block diagram of an exemplary method 304 of positioning TMS apparatus proximate one or more portions of a subject's brain as generally illustrated in the method of FIG. 2. The stimulation method 304 may include positioning TMS apparatus proximate one or more selected portions of the subject's brain 304. For example, the TMS apparatus may be positioned proximate the primary visual cortex 310, secondary visual cortex 312, primary auditory cortex 314, secondary auditory cortex 316, primary somatosensory cortex 318, and/or secondary somatosensory cortex 320. In one or more embodiments, the TMS apparatus may be positioned proximate all of these cortices (310-320) such that a subject may deliver TMS to all of the cortices (310-320) to, e.g., induce a meditation-like state of internally generated sensory information.

In one or more embodiments, positioning the TMS apparatus proximate one or more portions of the subject's brain 304 as shown in FIG. 3 may, e.g., temporarily disable the normal functioning of the primary visual, auditory, and somatosensory cortex which may then enable the higher association cortex to internally generate a construct of a sensory experience when the TMS apparatus is activated. The TMS may be performed during normal wakefulness, thereby simultaneously allowing the frontal lobes to function normally as during normal waking consciousness.

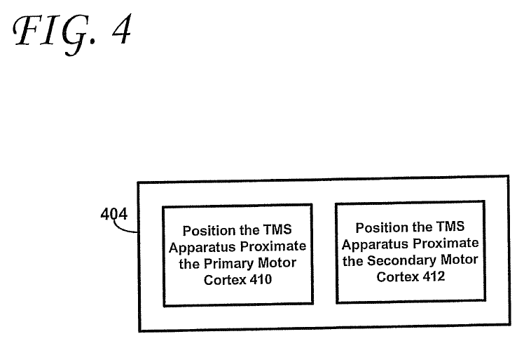

FIG. 4 is a detailed block diagram of another exemplary method of positioning TMS apparatus proximate one or more portions of a subject's brain as generally illustrated in the method of FIG. 2. The method 404 may include positioning TMS apparatus proximate one or more portions of the subject's brain. For example, the TMS apparatus may be positioned proximate the primary motor cortex 410 and the secondary motor cortex 412. In one or more embodiments, the TMS apparatus may be shaped in the form of an arch of TMS coils from ear to ear over the top of the head.

As described herein, the “primary motor cortex” may include Brodmann's areas 4, 6, 8, and/or, 44 and the “secondary motor cortex” may include Brodmann's areas 9, 10, 11, 45, 46, and/or 47. Further, both the “primary motor cortex” and the “secondary motor cortex” may include any other area/portion of a human's brain that may be involved in the processing of cortical motor control information as understood by one of ordinary skill in neurophysiology.

Further, in one or more embodiments, the TMS apparatus may be positioned proximate both motor cortices 410 & 412 such that TMS may be delivered to both motor cortices simultaneously to, e.g., inhibit normal neural processing activity of the primary and/or secondary motor cortices to induce temporary paralysis of a body part or limb, which may, when combined with the appropriate somatosensory stimulation, allow the block of physical sensation. Using such a method, less local and generalized anesthetic may be required for surgical procedures than may normally be required. However, it is noted that during a surgical procedure, non-localizable somatosensory pain information may still be processed at the thalamic level and, as such, some degree of sedation and/or analgesia may still be required.

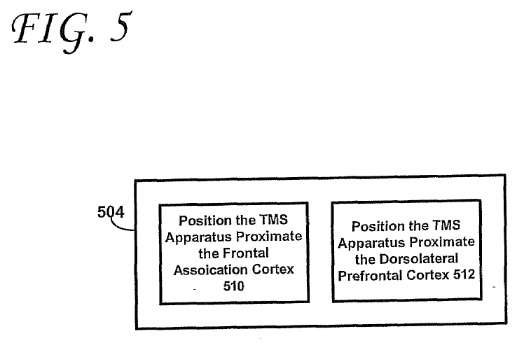

FIG. 5 is a detailed block diagram of another exemplary method of positioning TMS apparatus proximate one or more portions of a subject's brain as generally illustrated in the method of FIG. 2. The method 504 may include positioning TMS apparatus proximate one or more portions of the subject's brain. For example, the TMS apparatus may be positioned proximate the frontal association cortex 510 and the dorsolateral prefrontal cortex 512 (on each side of the frontal lobes).

As described herein, the “dorsolateral prefrontal cortex” may include Brodmann's areas 8, 9, 44, 45, 46, and/or 47 and/or any other area/portion of a human's brain that may be involved in the higher analytical, real time, and/or recent cognitive activity as understood by one of ordinary skill in neurophysiology.

Further, as described herein, the “frontal association cortex” may include Brodmann's areas 8, 9, 10, 44, 45, 46, and/or 47 and/or any other area/portion of a human's brain that may process cognitive activity in conjunction with the higher association cortex as understood by one of ordinary skill in neurophysiology.

In one or more embodiments, the TMS apparatus may be positioned proximate both of these cortices 510 & 512 such that TMS may be delivered to both cortices 510 & 512 simultaneously to, e.g., inhibit normal neural processing activity of the cortices 510 & 512 to induce the loss of waking consciousness. Such a method may be used in certain surgical procedures, or may also be employed during prolonged periods of virtual hibernation, such as, e.g., during manned space travel.

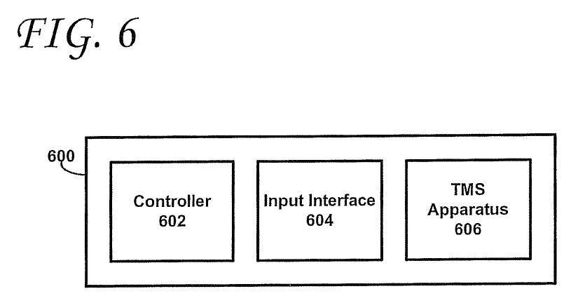

FIG. 6 is a diagrammatic representation of one exemplary stimulation system 600 according to the present invention. The stimulation system 600 may include controller 602, input interface 604, and TMS apparatus 606. The TMS apparatus may be similar to the TMS apparatus 110, 120, & 130 described herein with reference to stimulation system 100 depicted in FIG. 1.

The stimulation system 600 may further include a controller 602 that may be operably coupled (e.g., electrically) to the TMS apparatus 606. The controller 602 may be a microcontroller, or any other electrical controller, that may include inputs and outputs. At least one input of the controller 602 may receive data, e.g., an input signal, from the input interface 604.

The input interface 604 may be any interface by which a subject may control the stimulation 600. For example, the input interface 604 may be one or more buttons, one or more switches, one or more dials, a touch screen, etc. The input interface may be operably coupled (e.g., electrically) to the controller 602 such that a subject may use the input interface 604 to control the controller 602, which, in turn, may control the TMS apparatus 606. Using the input interface 604, a subject may choose which portions (i.e., the selected portions) of the subject's brain may receive TMS from the TMS apparatus 606. Also, using the input interface 604, a subject may select various parameters of the TMS to be delivered (or that is currently being delivered) to the subject. For example, a subject may be receiving an undesirable effect from the delivering of TMS at 0.8 teslas oscillating 30 hertz, and therefore, may desire to adjust the intensity to 1.4 teslas oscillating at about 6 hertz. Further, for example, a subject may desire TMS to be delivered to only a couple cortices, and therefore, may activate only the TMS a corresponding to the desires cortices. Further, using the input interface 604, the subject may adjust the duration of the stimulation to be delivered. For example, a subject may only want 15 minutes of stimulation, and therefore, may select only 15 minutes of stimulation from the stimulation system 600 using the input interface 604.

At least one output of the controller 602 may be coupled to the TMS apparatus 606 such that the controller may control aspects of the delivery of TMS to the subject. For example, the controller 602 may control various parameters of the TMS such as the time periods, field strength, frequency, morphology or rhythmic modulation, both regular and irregular, or various combinations of these parameters, as determined by principles of neurophysiology but may be tailored specifically to each subject, etc.

Although not shown, the stimulation system 600 may include a power source, e.g., a battery, line source (e.g., power cord), fuel cell, etc. Further, the controller 602, the input interface 604, and the TMS apparatus 606 may be a single, integral unit, or may be multiple interconnected units. Still further, the stimulation system 600 may be used in accordance with any method described herein including the systems and/or methods depicted in FIGS. 1-5.

The complete disclosure of the patents, patent documents, and publications cited in the Description of Exemplary Embodiments, and elsewhere herein are incorporated by reference in their entirety as if each were individually incorporated.

Illustrative embodiments of this invention are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A method of stimulating a subject's brain, the method comprising:
providing stimulation apparatus for delivering transcranial magnetic stimulation;
positioning the stimulation apparatus proximate one or more portions of the subject's brain, wherein the stimulation apparatus is located external to the subject's skull; and
delivering transcranial magnetic stimulation from the stimulation apparatus to the one or more portions of the subject's brain to inhibit normal neural processing activity in a selected portion of the subject's brain, whereby inhibiting the normal neural processing activity in the selected portion enables a release of a higher association cortex of the subject's brain to induce sensory information generation in the higher association cortex.

2. The method of claim 1, wherein the stimulation apparatus comprises an input interface, and wherein the method further comprises selecting the selected portion of the subject's brain using the input interface.

3. The method of claim 1, wherein the stimulation apparatus comprises an input interface, and wherein the method further comprises selecting one or more parameters of the transcranial magnetic stimulation to be delivered to the selected portion of the subject's brain using the input interface, wherein the parameters comprise at least one of:
field strength;
time period;
morphology;
rhythmic modulation; and
frequency.

4. The method of claim 1, wherein the transcranial magnetic stimulation delivered to the selected portion of the subject's brain oscillates at a frequency of about 0.1 hertz to about 250 hertz.

5. The method of claim 1, wherein the selected portion of the subject's brain comprises the primary visual cortex and/or the secondary visual cortex, and optionally wherein the higher association cortex generates visual sensory information.

6. The method of claim 1, wherein the selected portion of the subject's brain comprises the primary auditory cortex and/or the secondary auditory cortex, and optionally wherein the higher association cortex generates auditory sensory information.

7. The method of claim 1, wherein the selected portion of the subject's brain comprises the primary somatosensory cortex and/or the secondary somatosensory cortex, and optionally wherein the higher association cortex generates somatosensory sensory information.

8. A method of stimulating a subject's brain, the method comprising:
providing stimulation apparatus for delivering transcranial magnetic stimulation;
positioning the stimulation apparatus proximate the subject's primary motor cortex and secondary motor cortex, wherein the stimulation apparatus is located external to the subject's skull; and
delivering transcranial magnetic stimulation from the stimulation apparatus to the subject's primary motor cortex and secondary motor cortex to inhibit normal neural processing activity of the primary motor cortex and secondary motor cortex of the subject's brain, whereby inhibiting the normal neural processing activity of the primary motor cortex and secondary motor cortex induces temporary partial paralysis of the subject's body.

9. A method of stimulating a subject's brain, the method comprising:
providing stimulation apparatus for delivering transcranial magnetic stimulation;
positioning the stimulation apparatus proximate a selected portion of the subject's brain, wherein the selected portion of the subject's brain comprises at least one of the frontal association cortex and the dorsolateral prefrontal cortex, wherein the stimulation apparatus is located external to the subject's skull; and
delivering transcranial magnetic stimulation from the stimulation apparatus to the selected portion of the subject's brain to inhibit normal neural processing activity of the selection portion of the subject's brain, whereby inhibiting the normal neural processing activity of the selected portion of the subject's brain suppresses the subject's waking consciousness.

10. A stimulation system comprising:
stimulation apparatus for delivering transcranial magnetic stimulation, wherein the stimulation apparatus is positionable proximate two or more portions of a subject's brain, wherein the two or more portions of the subject's brain comprise two or more of the primary visual cortex, secondary visual cortex, primary auditory cortex, secondary auditory cortex, primary somatosensory cortex, and secondary somatosensory cortex, wherein the stimulation apparatus is located external to the subject's skull, wherein transcranial magnetic stimulation is deliverable from the stimulation apparatus to two or more selected portions of the two or more portions of the subject's brain to inhibit normal neural processing activity of the two or more selected portions to the subject's brain, whereby inhibiting the normal neural processing activity of the two or more selected portions of the subject's brain enables a release of a higher association cortex of the subject's brain to induce sensory information generation in the higher association cortex;
a controller electrically coupled to the stimulation apparatus, wherein the controller comprises an input interface to receive input from the subject, and wherein the controller controls the functionality of the stimulation apparatus; and
an input interface electrically coupled to the controller configured to receive input from the subject to control the stimulation apparatus.

11. The stimulation system of claim 10, wherein the transcranial magnetic stimulation apparatus comprises two or more coils, wherein each coil of the two or more coils are positionable proximate the subject's head.

12. The stimulation system of claim 10, wherein the input interface is configured to allow the subject to select the time period that the transcranial magnetic stimulation is to be delivered by the stimulation apparatus.

13. The stimulation system of claim 10, wherein the input interface is configured to allow the subject to adjust the field strength of the transcranial magnetic stimulation delivered by the stimulation apparatus.

14. The stimulation system of claim 10, wherein the input interface is configured to allow the subject to adjust the frequency of the transcranial magnetic stimulation delivered by the stimulation apparatus.

15. The stimulation system of claim 10, wherein the input interface is configured to allow the subject to adjust the rhythmic modulation of the transcranial magnetic stimulation delivered by the stimulation apparatus.

16. The stimulation system of claim 10, wherein the input interface is configured to allow the subject to adjust the morphology of the transcranial magnetic stimulation delivered by the stimulation apparatus.

17. The stimulation system of claim 10, wherein the input interface is configured to allow the subject to choose which portions of the two or more portions of the subject's brain receive transcranial magnetic stimulation from the stimulation apparatus.

18. The stimulation system of claim 10, wherein the stimulation system further comprises a power source, and wherein the stimulation system is a single integral unit.

19. The stimulation system of claim 10, wherein the stimulation apparatus is operable to deliver transcranial magnetic stimulation that oscillates at a frequency of about 0.1 hertz to about 250 hertz.

20. The stimulation system of claim 10, wherein the stimulation apparatus is operable to deliver transcranial magnetic stimulation having a magnetic field strength as measured at the subject's scalp of about 1 tesla or less.
